(19) United States
(12) Patent Application Publication
(10) Pub. No.: US 2013/0217083 A1
(43) Pub. Date: Aug. 22, 2013

(54) GERMINATING ZYGOSPORES OF UNICELLULAR, SPORE-FORMING ALGAE AS A SOURCE OF ABUNDANT EXTRACTABLE LIPID

(71) Applicant: ARIZONA BOARD OF REGENTS, acting for and on behalf of NORTHERN ARIZONA UNIVERSITY, (US)

(72) Inventor: Karen P. VanWinkle-Swift, Flagstaff, AZ (US)

(73) Assignee: ARIZONA BOARD OF REGENTS, acting for and on behalf of NORTHERN ARIZONA UNIVERSITY, Flagstaff, AZ (US)

(21) Appl. No.: 13/770,978

(22) Filed: Feb. 19, 2013

Related U.S. Application Data
(60) Provisional application No. 61/600,421, filed on Feb. 17, 2012.

(51) Int. Cl. C12P 7/64

(52) U.S. Cl. CPC C12P 7/64
USPC 435/134

(57) ABSTRACT

Described are procedures that promote the accumulation and release of intracellular lipid bodies from zygospores of single-celled alga. This disclosure describes procedures for manipulating the progression or synchronization of the algae's life cycle, and for simplifying lipid extraction by promoting spontaneous release of intracellular lipid reserves. The approach is relevant to the commercial production of biodiesel and food supplements derived from fatty acids.

### CROSS REFERENCE TO RELATED APPLICATION

This application claims priority to U.S. provisional application Ser. No. 61/600,421 filed Feb. 17, 2012, the entire disclosure of which is hereby incorporated by reference.

### STATEMENT OF FEDERALLY SPONSORED RESEARCH

Financial Assistance for this project was provided by the U.S. Government, NSF No. MCB-9728461 and NIH No. 1R15GM071374-02, and therefore the U.S. Government may own certain rights to this invention.

### BACKGROUND OF THE INVENTION

This invention relates to the production of lipids from algae, which lipids may be used, for example, as biodiesel and in food supplements.

### BRIEF SUMMARY OF THE INVENTION

This disclosure describes procedures that promote the accumulation and release of intracellular lipid bodies from zygospores of single-celled algae. An embodiment of the invention specifically described herein are procedures for the species Chlamydomonas monoica. This disclosure describes procedures for manipulating the progression or synchronization of the algae's life cycle, and for simplifying lipid extraction by promoting spontaneous release of intracellular lipid reserves. The approach is relevant to the commercial production of biodiesel and food supplements derived from fatty acids.

### DETAILED DESCRIPTION OF THE INVENTION

Described herein is a procedure for obtaining lipids from unicellular, spore-forming algae. An example of a specific genus from which the lipids may be obtained is Chlamydomonas, a common soil and water alga. Hundreds of species of this genus are known but only a few tend to be used routinely in laboratory research, for example: C. reinhardtii, C. eugametos, and C. monoica.

Environmental stress, particularly nitrogen starvation, typically results in limiting asexual reproduction (also referred to as vegetative growth) of algae, causing the algae to shift into sexual reproduction mode. During this shift, the vegetative cells differentiate into gametes. Generally speaking, there are two types of gametes, referred to as opposite mating types, although in some species the gametes may be referred to as male and female. In many Chlamydomonas species, they are referred to as mt⁺ (mating-type plus) and mt⁻ (mating-type minus). The gametes fuse, thereby producing zygotes. The zygotes are relatively transient, and mature into zygospores. Zygospores have walls that are very resistant to degradation.

The present invention involves certain procedures for synchronizing algae zygospore germination, and for harvesting zygospores during germination at the zygospore's most fragile stage, just prior to progeny release. The procedures further involve spore/cell lysis, release of lipid bodies into the surrounding culture medium, and harvesting the lipid bodies released by the lysed zygospore, as well as harvesting the lipid/oil-rich progeny cells.

More specifically, described herein are procedures wherein algae zygospores are subjected to synchronized germination in the presence of light, followed by inducing lysis of the zygospores during the germination process at a point prior to or substantially simultaneously with the release of progeny. When the zygospores are lysed, lipid bodies, as well as progeny containing lipid bodies, will be released, and are then harvested. The procedures described herein maximize the amount of lipids which can be obtained from algae.

The inventor has determined that the zygospores should preferably be lysed before they would naturally release progeny and return to vegetative growth, because vegetative growth will consume the lipid bodies. The goal is to avoid consumption of the lipid bodies by the progeny, in order to maximize the amount of lipid to be released from the zygospores and progeny, in a given culture/sample/batch, thereafter harvested. (Progeny cells are rich in lipid at the time of their release from the germinating zygospores. It is believed that when the progeny are released from zygospores in the absence of light, consumption of the lipid bodies by the progeny provides an energy reserve for survival in a non-dividing vegetative state in darkness.) The invention involves manipulating the rates of lipid consumption (lipid consumption results in undesirable reduction in lipid content) that normally follows the natural release of progeny cells from germinating zygospores (without induced lysis), by inducing germination under continuous illumination (for about 24-48 hours), thus allowing photosynthesis to provide the progeny with energy without the need for lipid consumption.

Some or all of the procedures described herein are believed to be utilizable with many different genera and species of algae, provided that the particular species in question accumulates sufficient lipids in its zygospore form, and the zygospore is able to be lysed during the germination process at a point prior to or substantially simultaneously with the release of the progeny cells from the zygospores. Certain parameters and conditions of the procedures described herein may require modification, depending upon the characteristics of the particular species used.

Chlamydomonas is an example of an algae genus with which the procedures described herein my be used. Examples of species within this genus which may be used are the C. reinhardtii and C. monoica species. In particular, C. monoica is preferred because lipid body formation occurs using CO₂ as the sole carbon source (i.e., without the requirement for an organic carbon source), and without the need to suppress starch synthesis.

An additional reason for preferring C. monoica over other Chlyamydomonas species is that the species is homothallic, meaning that cells of a single strain spontaneously differentiate into opposite mating types when grown in a nitrogen-deficient medium, resulting in zygospores that are derived from a single strain. In contrast, C. reinhardtii and all other well-studied Chlamydomonas species are heterothallic, meaning that zygospore formation requires interaction (i.e., mating) between strains of opposite mating types (referred to as “mating-type plus” and “mating-type minus”). Thus, zygospore formation in C. monoica requires maintenance and culturing of only a single strain of the alga rather than two.

Stress, and in particular nitrogen starvation, induces lipid accumulation in many alga. Lipid accumulation in some species, such as C. reinhardtii, requires an organic carbon source (for example, acetate). In other species, such as C. monoica, intracellular lipid is accumulated in response to nitrogen starvation but with no requirement for organic carbon. C. monoica does not, and cannot, use acetate as a carbon source to support growth in the absence of light. Instead, C. monoica is an obligate photoautotroph—an organism that requires light, water and CO₂ for synthesis of carbohydrates.

When environmental nitrogen reserves become limiting for growth, C. monoica (and other Chlamydomonas species) shift from asexual growth to sexual reproduction. The vegetative cells differentiate into gametes that fuse to produce transient zygotes. Zygotes then mature into heavily walled dormant zygospores, awaiting the return of more suitable environmental conditions.

The accumulation of lipids begins in the gametes and continues throughout maturation of the zygote into a dormant zygospore. The cytoplasm of a mature zygospore is comprised primarily of numerous, large lipid bodies that serve as an energy reserve. At the stage that the zygospore is mature, it appears to contain a larger amount of lipids than at any other point of the alga's lifecycle. When environmental conditions improve, such as the return of adequate inorganic nitrogen in the case of C. monoica, the zygospores germinate, releasing progeny cells that are capable of active vegetative growth as they consume the lipid reserves.

The zygospore is encased in a massive wall that is extraordinarily resistant to chemical and enzymatic degradation, making it difficult to access the lipids contained therein. Electron microscopy of mature zygospores indicates that the majority of the cytoplasm in the zygospores is lipid. In fact, as much as about 60% of the dry weight of a zygospore is extractable lipid. In the process described herein, the zygospores are lysed just prior to their release of progeny, resulting in the “release” of lipids from the alga. Such “released” lipids may then be harvested, for a variety of uses, such as biofuels, nutritional supplements, food additives, and the like.

The process of the invention generally involves the following steps:

1. Zygospore formation;

2. Purification of zygospores;

3. Washing and storage of zygospores;

4. Synchronization of zygospore germination; and

5. Lysing of zygospores to release lipids and/or harvesting of lipid-rich progeny cells.

The following describes a process according to the invention for producing and obtaining lipids from zygospores.

1. Zygospore Formation. Zygospore formation is induced. For example, zygospore formation may be induced by transferring vegetative algae cells (grown in liquid or on an agar surface) to a nutrient-deprived medium.

For example, C. monoica is induced to form zygospores by transferring to a nitrate- and phosphate-depleted liquid medium. Mating occurs about 36-48 hours after transfer, with mature zygospores present within about 5-7 days after transfer. For this species, mating efficiency was about 60-75% at about 20° C., but dropped to about 1-20% at about 24-25° C. Different mating temperatures may be optimal for other species. The term “mating efficiency” refers to the ratio of individual alga cells that have mated in the culture compared with the pre-mating population (i.e., the vegetative alga cells).

The size of mating cultures may be scaled up by aeration, such as by bubbling purified air through the culture. The term “size of mating culture” means the amount of vegetative alga cells that are caused to mate. In addition, or as alternative, to aeration, one can increase the surface to volume area ratios of the mating culture, in order to scale up the size of the mating culture without compromising the mating efficiency. One can maximize the surface to volume ratio by using only sufficient liquid media to cover the alga and prevent it from drying out during aeration. Increasing the surface to volume ratio of the mating culture has the added benefit that less culture media is required, which ultimately has the benefit of less effort required to separate the alga from the media when the lipids are ultimately recovered therefrom.

2. Purification of Zygospores. Mature zygospores are preferably purified (e.g., separated from unmated vegetative cells). In an exemplary embodiment of the invention, the mature zygospores are purified by layering the culture (of zygospore cells and vegetative cells) onto the surface of a “cushion” of 40% sucrose in a centrifuge tube. Instead of sucrose, a saturated salt solution such as 35% NaCl may be utilized, although a 40% sucrose solution is preferred because it retains the viability of the zygospores better.

Centrifugation provides a rapid way to separate the zygospores from the vegetative cells. After centrifugation at about 3,000×g for about 5 minutes, only the zygospores remain on the surface of the sucrose layer. Although centrifugation provides a more rapid separation means, it may result in damage to cell morphology and/or viability. Such adverse effects of centrifugation may possibly be avoided by centrifuging for the shortest time possible, or use or a refrigerated centrifuge, or chilling the zygospores prior to centrifuging. Instead of centrifugation, the zygospores and vegetative cells (unmated cells) may be permitted to spontaneously separate (from one another) over a period of several hours. In such case, the zygospores are separated from the unmated cells due to differences in their respective buoyancy.

The separated zygospores are easily removed by pipetting or another method, such as siphoning. On a larger scale application, a siphon could be positioned near the bottom of the sucrose layer in the vessel containing the zygospores and vegetative cells, and the sucrose layer removed (such as by draining) from the bottom of the vessel.

3. Washing and Storage of Mature Zygospores. Mature zygospores are washed. The washed zygospores may optionally be stored after washing. Preferably, the water for washing and storage is distilled water or water that has been subjected to reverse osmosis. The viability of zygospores remains high even after one (1) year of storage under refrigeration and in darkness. The washing water is chilled or at room temperature. In an exemplary embodiment of the invention, the water is less than about 30° C.

Washing removes residual sucrose; the presence of sucrose may interfere with further processing of the zygospores due to its stickiness, and also because sucrose might undesirably serve as a food source for vegetative cells and/or for microbial contaminants (if any). The washing water is preferably water that has been subjected to reverse osmosis. Even more preferably, the washing water is distilled water. Most preferably, the washing water is sterile, distilled water.

The storage water is preferably water that has been subjected to reverse osmosis. Even more preferably, the washing water is distilled water. Most preferably, the washing water is sterile, distilled water.

It is preferred that the washing water and storage water are each free of microbial contaminants and chemicals which may be toxic to the alga or which might promote premature germination, or which might otherwise interfere with the process of the invention.

Storage in darkness (no light) is preferred to maintain viability of the zygospores. Further, for C. monoica and perhaps other species, it is preferable that storage temperatures not exceed about 25° C.

Zygospores stored in the light at room temperature (about 20-25° C.) for two (2) months exhibited significant loss of viability and also took on a bleached appearance, as opposed to the normal green color of healthy zygospores. In contrast, zygospores stored in the dark at room temperature (about 20-25° C.) for two (2) months retained high levels of viability and retained the normal healthy green color.

Zygospores stored in the light for two (2) months at room temperature (at about 20-25° C.) demonstrated 0 (zero) % germination, whereas zygospores stored in darkness for two (2) months under refrigeration (at about 4° C.) demonstrated about 98% germination.

In an exemplary embodiment, the zygospores are stored at a temperature in the range of about 20-25° C. in the absence of light. In another embodiment, the zygospores are stored under refrigeration (at about 4° C.).

4. Synchronization of Zy₂ospore Germination.

Efficiency of zygospore germination refers to the ratio of zygospores in a particular sample or batch that germinate, to the total number of zygospores in that sample or batch. It is a goal of the process of this invention to increase the efficiency of zygospore germination.

Synchronization of zygospore germination refers to causing substantially all of the zygospores in a particular sample or batch to germinate substantially simultaneously. It is a goal of the process of this invention to provide synchronization of zygospore germination.

The process of the invention enhances the efficiency and synchronization of zygospore germination. It appears that the speed of germination, as well as the germination efficiency and germination synchronicity can be enhanced by storing the zygospores, as compared to freshly collected zygospores. Storage of the zygospores for at least 3-4 weeks appears beneficial to enhancing germination efficiency and synchronization.

Freshly collected zygospores germinate more slowly and less synchronously than stored zygospores. Storage for about 3-4 weeks appears necessary to shift the preferred requirements for and the timing/synchronization of germination. In the case of C. monoica, for fresh zygospores (removed from the mating culture at 7 days, purified and washed, and then plated for germination) the best and most synchronous germination occurs on medium containing NH₄Cl as the nitrogen source (HS medium). Furthermore, the zygospores should be plated and then held in darkness for 3-5 days before transferring the plates to continuous illumination. Once illuminated, germination (release of progeny cells) begins about 24-30 hours later and is maximal by about 48 hours after light induction (at about 20° C.).

Storage in darkness significantly decreases loss of viability and reduction in germination efficiency and synchronization. Zygospores stored refrigerated for about one (1) year in darkness have been found to have about a 95% germination rate. Zygospores stored for several years in the dark and under refrigeration have been found to remain viable as long as they are kept moist.

In a preferred embodiment of the invention, the zygospores are stored in darkness for at least 3-4 weeks at room temperature. In yet another preferred embodiment, the zygospores are stored in darkness for at least 3-4 weeks under refrigeration. It takes about 3-4 weeks of storage without nutrients before the zygospores will rapidly germinate when nutrients and light are returned.

To synchronize zygospore germination, the previously stored zygospores are exposed to nutrients, such as by being plated on standard inorganic medium (agar-solidified). Alternatively, the zygospores may be placed on a porous membrane, which is itself placed on top of a fine-grained, highly purified sand substrate saturated with standard culture medium. Non-limiting examples of porous membranes that may be used include cellulose acetate, nitrocellulose and nylon membranes. The porous membrane typically will have pores ranging in size from about 0.22 to 1.0 μM.

If the zygospores are fresh and/or have not been stored for at least three (3) weeks, they should be placed in darkness for 1-7 days in a nutrient medium. (A period closer to the lower end of this range is used if the zygospores have been stored previously for more than a few days. The “fresher” the zygospores are, i.e., the less time that has elapsed since they were formed, the more days in darkness in nutrient medium that are required.) The zygospores are then exposed to light continuously throughout the germination process (and thereafter). Germination occurs about 24-36 hours after light induction at about 20° C., although zygospores may continue to germinate for an additional 12 hours or so.

For C. monoica zygospores stored refrigerated (about 4° C.) for three weeks or longer, the preferred medium for germination contains NaNO₃ as the nitrogen source (BM). The zygospores plated on BM are placed immediately under continuous illumination. Germination begins around 24-30 hours later and is maximal by 48 hours. As above, the synchrony is limited with more zygospores continuing to germinate during the 12 hour period. A primary advantage of using the stored zygospores is the elimination of the requirement for a dark incubation period after plating. In fact, dark incubation of aged zygospores after plating should be avoided as it leads to premature germination of the zygospores (and undesirable consumption of lipid) during the dark incubation period.

As noted above, freshly collected C. monoica zygospores germinate more efficiently on HS (ammonium-containing) medium than on BM (nitrate-containing) medium. In fact, overall germination of freshly collected zygospores on BM medium is poor even after prolonged periods of exposure to light. In contrast, zygospores stored in refrigeration (4° C.) for at least 3 weeks germinate efficiently on either HS or BM medium, while zygospores that have been stored for several months germinate most efficiently on BM.

Although we have found a negative correlation between temperature and mating of C. monoica, the germination of C. monoica zygospores occurs more rapidly at 25° C. than at 20° C. Using younger zygospores, HS medium, and a brief dark incubation period after plating, the germination of spores begins within 18 hours after transfer to the light and is near maximal by 24 hours. Using the older stored zygospores, BM medium and a continuous light protocol, the spores begin to germinate within 24 hours and are near maximal by 32 hours in the light. In contrast to the improved germination at 25° C. (versus 20° C.) a reduction in germination efficiency occurs at 27° C.

The speed of germination and germination synchronicity is better on medium solidified with gelrite rather than agar. It is not presently known with certainty whether this is due to a stimulating effect of gelrite or an inhibitory effect of the agar, although it is believed that this may be due to inhibitory contaminants in the agar.

In scaled-up, commercial embodiments of the invention, it is anticipated that instead of the media discussed above, moistened sand beds overlain with porous artificial membranes may be substituted as the substrate for zygospore germination. More specifically, sand that has been washed with water obtained via reverse osmosis or distilled water should be used. The sand should then be moistened with the desired culture medium, such as HS or BM or the like). Preferably, relatively clean sand is used. Non-limiting examples of sand that may be used include builder's sand, Reptisand® aquarium sand, silicon dioxide, and the like. The porous membrane may be comprised of any material that will assist in promoting germination, such as nylon, cellulose acetate, nitrocellulose, Metricel®, or Supor® (Metricel® and Supor® are offered by Pall Life Sciences.). The foregoing membrane materials have pore sizes in the range of about 0.22-1.0 micron, but other pore sizes may be effective as well. It was found that pore sizes affected the amount of spreading of zygospores that occurred but did not appear to affect overall germination efficiency, speed or synchronicity.

5. Lysing of Zygospore to Release Lipids. Just prior to the release of progeny, the zygospore is very susceptible to lysis. Simply touching the zygospore results in lysis of progeny cells unless one waits until natural release of the cells has occurred. Even washing cells from the plates at this fragile stage is a sufficient disturbance to cause cell lysis and the release of intracellular lipid bodies. The invention contemplates the use of a variety of disruption techniques for lysis of progeny cells and release of lipid bodies (e.g., stirring or agitation via centrifugation, osmotic shock, vortexing of cells in the presence or absence of glass beads, sonication, freeze/thawing, or abrasion). Some of the foregoing lysis methods are more effective than others.

In a preferred embodiment, the zygospores are subjected to vortexing. More specifically, it has been found that vortexing for about 60 seconds in the presence of 2 mm diameter glass bead results in breakage of at least 75% of the zygospores in the sample. The beads should be at least 1 mm, and preferably 2 mm in diameter.

The volume of culture to the volume of glass beads influenced the results. The best breakage results occurred when the liquid (the zygospores in the medium) just reaches the top layer of beads.

After breakage via vortexing, the zygospores are subjected to extraction. In a preferred embodiment, extraction is accomplished using hexane, but other methods and substances known to one of ordinary skill in the art may be used for the extraction step.

Many environmental factors, as well as the genetic background of the strain of C. monoica or other species being used, affect the timing and synchrony of germination. For example, the longer the zygospores are stored (in water under refrigeration), the less time they will need to be held in darkness after the return of nutrients (but before the light induction). It is believed that with appropriate choice of storage time (refrigerated) it may be possible to induce efficient germination by transferring the zygospores directly to continuous illumination simultaneously with the addition of an appropriate nitrogen source.

The following are environmental factors determined by the inventor to interact, affecting both synchrony of germination and overall efficiency: (1) age of the zygospores (i.e., how long in cold storage after collection); (2) how long the zygospores have received nutrients but not light prior to induction (i.e., the time separating the transfer of zygospores onto standard medium at room temperature and transfer of the zygospores to continuous illumination); (3) nature of the plating medium (e.g., nitrate or ammonium as the nitrogen source) during the pre-induction period in the dark and subsequent light induction (the spores remain on the same medium when they are shifted into the light); and (4) temperature at the time of light induction (after dark incubation, and maintained throughout the germination process).

In general, zygospores that have been stored refrigerated in water for more than about two (2) weeks require shorter darkness (pre-light) incubation times than do fresher zygospores to promote the most efficient and synchronous germination. Zygospores refrigerated for more than about two (2) weeks require about 0-3 days in the dark on standard media, whereas zygospores refrigerated for less than about two (2) weeks require somewhat longer periods (about 5-7 days).

It is believed that raising the temperature from about 20° C. to about 24 ° C. during light induction increases the speed and synchrony of germination in “old” stored zygospores (i.e., in zygospores that are more than about two (2) weeks old).

The speed of germination can be increased in fresh zygospores by increasing the length of the dark incubation period to about 5-7 days.

Zygospores stored refrigerated in water for about two (2) weeks or more will show premature germination in the dark (within about three (3) days at room temperature), which is undesirable and should be avoided. Premature germination appears to be avoidable by using a standard medium (called BM) that contains sodium nitrate as the nitrogen source, although this is less effective if the spores are very old and held in darkness on the BM medium for more than seven (7) days. However, if a standard medium (called HS) that contains ammonium chloride as the nitrogen source is used, there is a high likelihood of premature germination.

A variety of substrates may be used, provided that they promote efficient zygospore germination and facilitate recovery of released lipid. Germination of spores has been accomplished on membrane filters placed on standard agar solidified medium, as well as on foam “platforms” floating on standard liquid medium. Sand, particularly highly purified, fine-grained sand, is an alternative substrate when saturated with standard aqueous culture medium.

Zygospores stored refrigerated for several weeks or more prior to plating on agar will germinate in the dark within about three (3) days. Longer storage results in more rapid germination. It is believed that that spore viability is reduced if spores are either dried (desiccated) or frozen. Zygospores stored (refrigerated) as moist pellets continue to show 75-100% viability after six (6) months. It is believed that zygospores stored at room temperature will also retain high viability if they remain moist.

The following temperature parameters are preferred. The storage temperature for zygospores refrigerated in water is about 4° C. The temperature for dark incubation prior to light induction is “room temperature”, which is usually about 23-25° C. The standard temperature for light induction (and beyond) is about 20-25° C.

The following are examples of strains of C. monoica which were evaluated. Storage time, pre-induction dark induction period, and induction culture medium have been tested for effects on germination in the original 22B and U220 strains; germination is enhanced on medium solidified with gellan gum as compared to agar, preferred culture medium is determined by age of zygospores (storage time); longer dark incubation promotes more rapid and synchronous germination; 22B germinates slightly more rapidly and is less affected by culture medium composition (but zygospore morphology is poor). (Thus far, 80-100% germination can be achieved with the original 22B and U220 strains at 20° C., however speed of onset and synchronization might be improved by raising the temperature).

Subclone 98 of U220 has demonstrated an enhanced mating efficiency (50-60%) as compared with U220 (<20%).

### Technical Details of an Exemplary Embodiment of the Invention

Strain maintenance: The wildtype strain (U220) of Chlamydomonas monoica is maintained on agar-solidified standard minimal medium containing ammonium chloride as the nitrogen source (referred to as HS medium) under continuous cool white fluorescent illumination at 20° C. The strain is transferred to fresh medium every two weeks and is sub-cloned periodically to maintain high mating potential.

Induction of mating and zygospore formation: Vegetative cells are removed from the HS agar plates and are re-suspended in a low nitrogen, low phosphate liquid medium (referred to as LPN) containing 50 mg/L NaNO₃, 7.5 mg/L K₂HPO₄ and 17.5 mg/L KH₂PO₄. Mating efficiency is maximized if the starting cell density in LPN is 0.5-1.0×10⁶ cells/mL. We routinely induce mating in small volume cultures (1.0 mL LPN in 12×75 mm glass tubes). The tubes containing the cell suspension are capped with foam plugs and rested nearly horizontal (in transparent plastic trays) to maximize surface area/gas exchange. Recently we have been able to scale up to a 100 mL culture held in a 500 mL flask by including continuous bubbling with filtered air delivered through a glass pipette inserted through the foam plug closure and extending just below the surface of the liquid. Under continuous illumination from cool white fluorescent lamps (30 -75 μE of photosynthetically active radiation) and incubation at 20° C., mating occurs within 24-36 hours after transfer to LPN medium. Zygospores are fully mature (with extensive lipid accumulation) within seven days.

Harvesting of mature zygospores: Under the culture conditions described above, a seven-day-old LPN culture typically will be comprised of 50% zygospores. The zygospores can be separated from unmated cells by carefully layering the cell suspension on a “cushion” of aqueous 40% sucrose or 35% NaCl followed by centrifugation at 3,000×g for 5 minutes. After centrifugation, the zygospores remain above the sucrose/salt layer and can be removed easily by pipetting. The zygospores are then washed twice by re-suspension and centrifugation in distilled water. The purified zygospores are stored at 4° C. in a small volume of distilled water to prevent desiccation and can be maintained for months, or even years, if kept moist.

Induction of zygospore germination: A dense suspension of purified zygospores is plated onto standard agar-solidified HS medium and the spores are incubated at 20-25° C. in darkness for 3-7 days. The plated spores are then transferred to continuous illumination (30-75 μE continuous cool white fluorescent light). Semi-synchronous spore germination (release of meiotic progeny cells) occurs 20-48 hours following the transfer to light. Under the above conditions, the efficiency of zygospore germination is typically 85-100%. Zygospores can also be germinated on the surface of porous cellulose or nylon membranes (0.22-0.45 μm pores) placed on standard agar or on porous foam platforms floating on HS liquid medium.

Induction of cell lysis and release of stored lipid bodies: When the zygospores begin to swell and the first evidence of spore germination is observed (about 20 hours after light induction), a small volume of distilled water is added to the agar or membrane surface and the zygospores are mixed into suspension with a glass rod. This disturbance is sufficient to cause lysis of swollen zygospores and their immature wall-less progeny cells, with release of the lipid bodies into the suspension medium.

The following are several advantages of the process of the invention:

Zygospores may provide the richest source of lipid in terms of percent lipid/cell biomass, and may be stored for years, if not decades, without loss of viability due to the extreme resistance of the zygospore wall to digestion or chemical degradation.

Germination can be synchronized in large populations of zygospores by manipulation of environmental conditions. Germination of zygospores includes the natural breakdown of the zygospore wall as well as the distribution of lipids to progeny cells that are initially naked (without cell walls). This eliminates the need for enzymatic or chemical digestion of the wall prior to lipid extraction. Zygospore germination can be induced en masse on a moist surface without the need for large scale liquid culturing (e.g., artificial ponds).

In populations of zygospores that have been well-synchronized using the procedures described herein, lipid bodies can be released from the majority of progeny cells by simply disturbing the zygospores at their most fragile stage, when the unreleased progeny cells are still “naked,” i.e., they have not formed their cell walls. If not disturbed, zygospores will release the progeny cells (which remain lipid-rich) and can be harvested for oil extraction before consumption of the lipids occurs.

In summary, the invention relates to a method for obtaining lipids from algae, comprising synchronizing germination of zygospores in a culture of algae cells, lysing the zygospores just prior to release of progeny cells from the zygospores, and harvesting lipids released from the zygospores. In addition to harvesting the lipids released from the zygospores when the zygospores are lysed, lipids may also be harvested from the progeny cells that are released from the zygospores.

The method comprises the following steps: (a) forming zygospores of the algae; (b) purifying the zygospores; (c) washing the zygospores; (d) storing the zygospores; (e) synchronizing zygospore germination; (f) lysing the zygospores, just prior to release of progeny cells, to release lipids; and (g) recovering the lipids. The lipids that are recovered or harvested are those released from the lysed zygospore, but in addition, lipids present in the progeny cells can also be recovered or harvested.

The zygospores are formed by subjecting vegetative algae cells to stress, such as lack of nutrients.

More particularly, the method used to synchronize germination of zygospores will vary based upon how much time has passed since the zygospores were formed. If the zygospores are less than about 3 weeks old, then the zygospores must first be maintained in a nutrient medium in darkness for 1 to 7 days, and then exposed to continuous light.

If the zygospores are more than about 3 weeks old and have been maintained without a nutrient solution in darkness for those at least 3 weeks, then instead of maintaining the zygospores in a nutrient medium for 1 to 7 days, the zygotes are substantially simultaneously provided with a nutrient medium and continuous light in order to synchronize germination of the zygospores. Preferably, the zygospores are maintained in darkness without nutrients at a temperature in the range of about 4° C. to about 25° C.

Preferably, the zygospores are stored by maintaining them in darkness for at least about 3 weeks. More preferably, the zygospores are stored in darkness at a temperature in the range of about 4° C. to about 25° C. Assuming that the zygospores have been stored for at least about 3 weeks, the zygospores are then exposed substantially simultaneously to nutrients and substantially continuous light. This exposure may be accomplished by placing the zygospores on a membrane placed on a sand bed.

More specifically described herein is an exemplary embodiment of the method for obtaining lipids from Chlamydomonas monoica algae, said embodiment comprising the following steps:

(a) forming zygospores from the algae in a vegetative state by depriving it of nitrate and phosphate;

(b) purifying the zygospores by separating the zygospores from other cells;

(c) washing the zygospores;

(d) storing the zygospores for about 3 to about 4 weeks in darkness without nutrients at a temperature in the range of about 4° C. to about 25° C.;

(e) synchronizing zygospore germination by substantially simultaneously providing the zygospores with a nutrient medium containing nitrate and phosphate and subjecting the zygospores to substantially continuous light;

(f) lysing the germinated zygospores, just prior to release of progeny cells, to release lipids; and

(g) recovering the lipids.

What is claimed is:

1. A method for obtaining lipids from algae, comprising
(a) synchronizing germination of zygospores in a culture of algae cells,
(b) lysing the zygospores just prior to release of progeny cells from the zygospores, and
(c) harvesting lipids released from the zygospores.

2. The method of claim 1, wherein step (c) further comprises harvesting lipids from the progeny cells released from the zygospores.

3. The method of claim 1, wherein step (a) further comprises maintaining the zygospores in a nutrient medium.

4. The method of claim 3, wherein step (a) further comprises maintaining the zygospores in a nutrient medium in darkness, and then exposing the zygospores to continuous light.

5. The method of claim 4, wherein the zygospores in a nutrient medium are maintained in darkness for 1 to 7 days.

6. The method of claim 1, wherein step (a) further comprises maintaining the zygospores in darkness for at least about 3 weeks, and then providing to the zygospores a nutrient medium and continuous light.

7. The method of claim 6, wherein step (a) comprises maintaining the zygospores in darkness without nutrients at a temperature in the range of about 4° C. to about 25° C.

8. The method of claim 1, wherein the algae is of the genus Chlamydomonas.

9. The method of claim 8, wherein the algae is Chlamydomonas monoica.

10. A method for obtaining lipids from algae, comprising the following steps:
(a) forming zygospores of the algae;
(b) purifying the zygospores;
(c) washing the zygospores;
(d) storing the zygospores;
(e) synchronizing zygospore germination;
(f) lysing the zygospores, just prior to release of progeny cells, to release lipids; and
(g) recovering the lipids.

11. The method of claim 10, wherein step (g) further comprises recovering lipids from the progeny cells.

12. The method claim 10, wherein the algae is of the genus Chlamydomonas.

13. The method of claim 12, wherein the algae is Chlamydomonas monoica.

14. The method of claim 10, wherein step (a) further comprises forming zygospores by subjecting vegetative algae cells to stress.

15. The method of claim 13, wherein in step (d) storing comprises maintaining the zygospores in darkness for at least about 3 weeks.

16. The method of claim 15, wherein in step (d) storing further comprises maintaining the zygospores at a temperature in the range of about 4° C. to about 25° C.

17. The method of claim 16, wherein step (e) further comprises maintaining the zygospores in darkness without nutrients at a temperature range of about of about 4° C. to about 25° C., and then exposing the zygospores to nutrients and substantially continuous light.

18. The method of claim 17, wherein in step (e) the step of exposing the zygospores to nutrients in accomplished by placing the zygospores on a membrane placed on a sand bed.

19. A method for obtaining lipids from Chlamydomonas monoica algae, comprising the following steps:
(a) forming zygospores from the algae in a vegetative state by depriving it of nitrate and phosphate;
(b) purifying the zygospores by separating the zygospores from other cells;
(c) washing the zygospores;
(d) storing the zygospores for about 3 to about 4 weeks in darkness without nutrients at a temperature in the range of about 4° C. to about 25° C.;
(e) synchronizing zygospore germination by substantially simultaneously providing the zygospores with a nutrient medium containing nitrate and phosphate and subjecting the zygospores to substantially continuous light;
(f) lysing the germinated zygospores, just prior to release of progeny cells, to release lipids; and
(g) recovering the lipids.
